(19) United States
(12) Patent Application Publication
(10) Pub. No.: US 2012/0041311 A1
(43) Pub. Date: Feb. 16, 2012

(54) AUTOMATED THREE DIMENSIONAL ACOUSTIC IMAGING FOR MEDICAL PROCEDURE GUIDANCE

(75) Inventor: Anthony M. Gades, Snohomish, WA (US)

(73) Assignee: KONINKLIJKE PHILIPS ELECTRONICS N.V., EINDHOVEN (NL)

(21) Appl. No.: 13/140,051

(22) PCT Filed: Dec. 7, 2009
(86) PCT No.: PCT/IB09/55560

§ 371 Date: Jun. 16, 2011

Related U.S. Application Data
(60) Provisional application No. 61/140,251, filed on Dec. 23, 2008.

(51) Int. Cl. A61B 8/14

(52) U.S. Cl. 600/443

(57) ABSTRACT

A system and method of three dimensional acoustic imaging for medical procedure guidance includes receiving (410) an acoustic signal that is scanned to interrogate a volume of interest; determining (430) a location of a procedural device within the interrogated volume from the acoustic signal; and displaying (470) on a display device (130) a first view of a first plane perpendicular to an orientation of the procedural device. Beneficially, a second view of at least one plane perpendicular to the first plane is also displayed. Also beneficially, a third view of a third plane perpendicular to the first plane and to the second plane is also displayed. Also beneficially, the first, second, and third views are displayed at the same time.

This invention pertains to acoustic imaging apparatuses and methods, and more particularly to an acoustic imaging apparatus and method with automatic three dimensional imaging for medical procedure guidance.

Acoustic waves (including, specifically, ultrasound) are useful in many scientific or technical fields, such as in medical diagnosis and medical procedures, non-destructive control of mechanical parts and underwater imaging, etc. Acoustic waves allow diagnoses and visualizations which are complementary to optical observations, because acoustic waves can travel in media that are not transparent to electromagnetic waves.

In one application, acoustic waves are employed by a medical practitioner in the course of performing a medical procedure. In particular, an acoustic imaging apparatus is employed to provide images of a volume of interest to the medical practitioner to facilitate successful performance of the medical procedure. In particular, acoustic images can be employed by the medical practitioner to guide a procedural device toward a target area where the procedural device is to be employed.

One example of such an application is a nerve block procedure. In this case, the medical practitioner guides an anesthesia needle toward a nerve where the blocking agent is to be injected. Other examples include procedures involving a radiofrequency ablation (RFA) needle, a biopsy needle, cyst drainage, catheter placement, line placement, etc.

For such acoustic imaging procedural guidance, it is desirable to allow the practitioner to see the procedural device and easily visualize its location, orientation, and trajectory with respect to a target area where the device is to be employed. In conventional arrangements this is not always possible because the procedural device may not be precisely aligned with the scan plane of the acoustic transducer and in this case, it cannot be imaged. Additional complications in visualizing the procedure device can occur when a device like a needle bends or deflects as it is being inserted.

Other medical procedures can suffer from similar problems in the employment of acoustic imaging during the procedure.

Accordingly, it would be desirable to provide an acoustic imaging apparatus that can more easily allow a medical practitioner to visualize the location, orientation, and trajectory of a procedural device with respect to a target area where the device is to be employed.

In one aspect of the invention, an acoustic imaging apparatus comprises: an acoustic signal processor adapted to process an acoustic signal that is scanned to interrogate a volume of interest and is received by an acoustic transducer; a display device for displaying images in response to the processed acoustic signal; a control device that is adapted to allow a user to control at least one operating parameter of the acoustic imaging apparatus; and a processor configured to determine a location of a procedural device within the interrogated volume from the processed acoustic signal, wherein acoustic imaging apparatus is configured to display on the display device a first view of a first plane perpendicular to an orientation of the procedural device.

In another aspect of the invention, a method of three dimensional acoustic imaging for medical procedure guidance comprises: receiving an acoustic signal that is scanned to interrogate a volume of interest; determining a location of a procedural device within the interrogated volume from the acoustic signal; and displaying on a display device a first view of a first plane perpendicular to an orientation of the procedural device.

In yet another aspect of the invention, a second view of a second plane perpendicular to the first plane is also displayed.

In a further aspect of the invention, a third view of a third plane perpendicular to the first and second planes is also displayed.

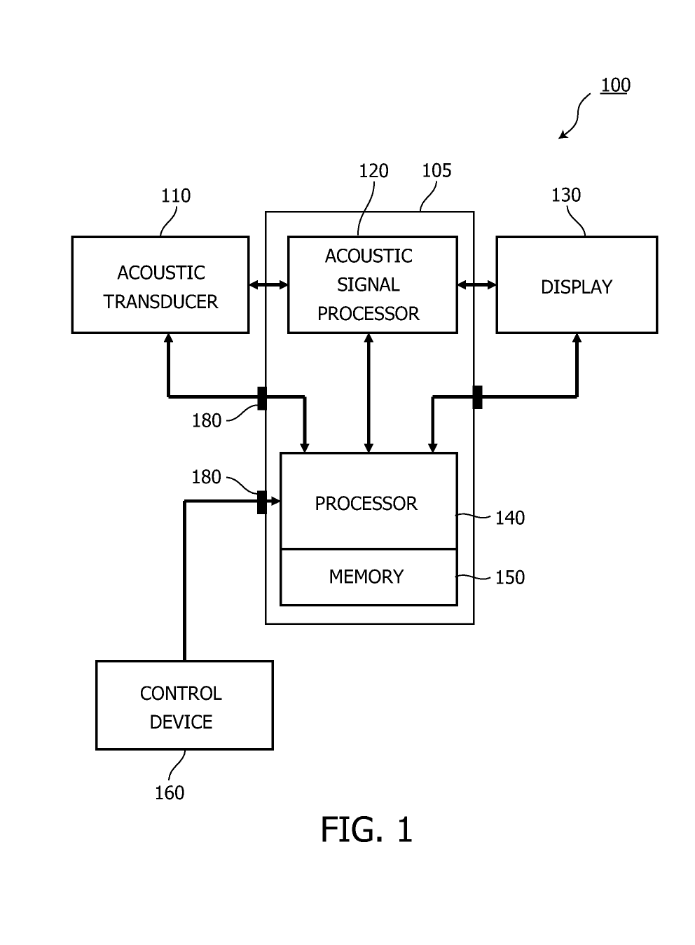

FIG. 1 is a block diagram of an acoustic imaging device.

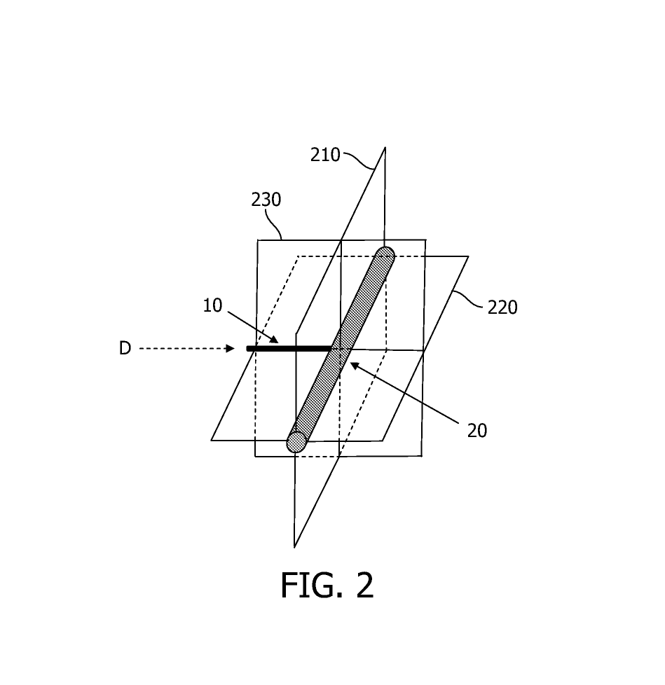

FIG. 2 illustrates an exemplary arrangement of three planes with respect to a procedural device and a body part toward which the procedural device is being directed.

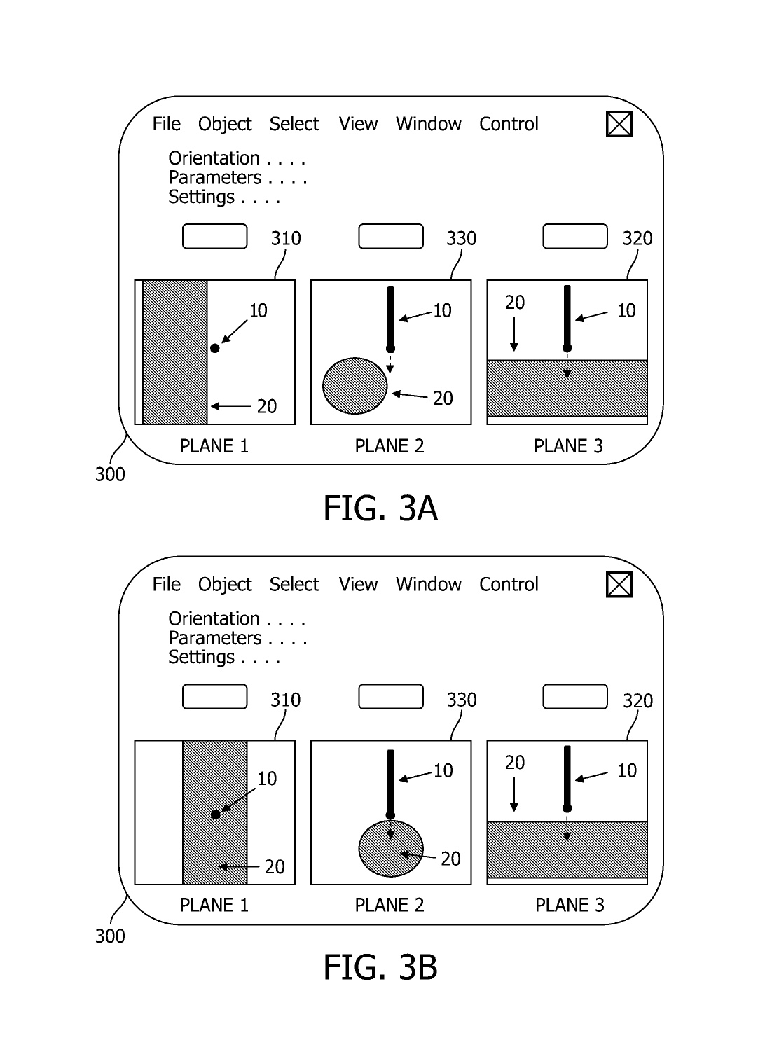

FIG. 3A illustrates a display of the three planes shown in FIG. 2 according to a first example.

FIG. 3B illustrates a display of the three planes shown in FIG. 2 according to a second example.

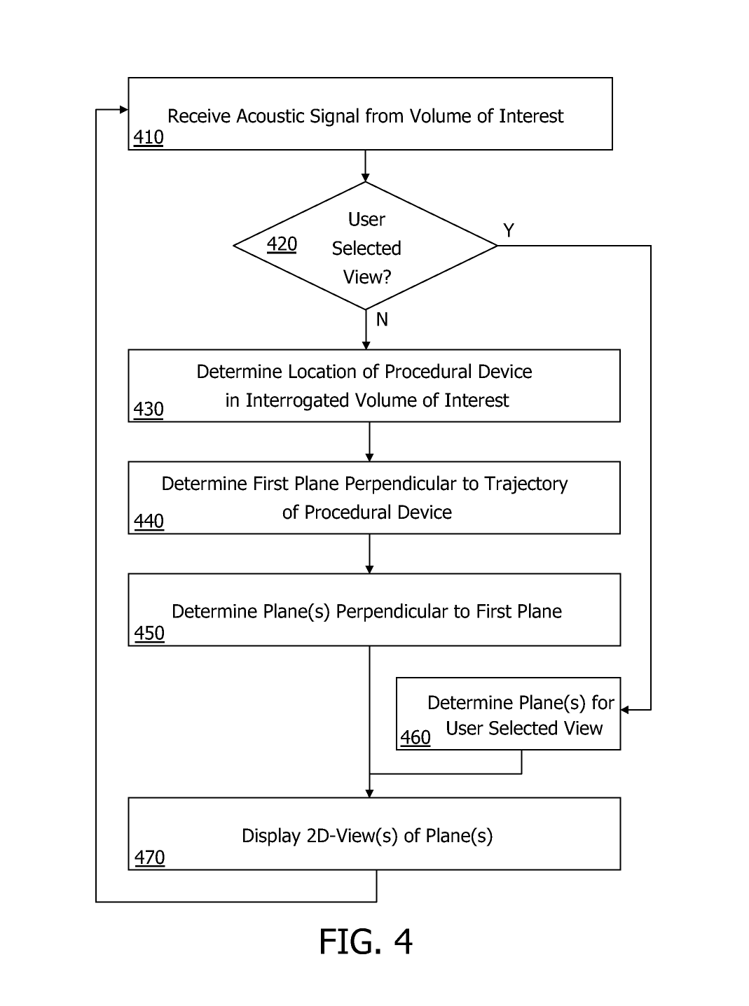

FIG. 4 illustrates a flowchart of a method of three dimensional acoustic imaging for medical procedure guidance.

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

FIG. 1 is a high level functional block diagram of an acoustic imaging device 100. As will be appreciated by those skilled in the art, the various “parts” shown in FIG. 1 may be physically implemented using a software-controlled microprocessor, hard-wired logic circuits, or a combination thereof. Also, while the parts are functionally segregated in FIG. 1 for explanation purposes, they may be combined in various ways in any physical implementation.

Acoustic imaging device 100 includes an acoustic (e.g., ultrasound) transducer 110, an acoustic (e.g., ultrasound) signal processor 120, a display device 130, a processor 140, memory 150, and a control device 160.

In acoustic imaging device 100, acoustic signal processor 120, processor 140, and memory 150 are provided in a common housing 105. However, display device 130 may be provided in the same housing 105 as acoustic signal processor 120, processor 140, and memory 150. Furthermore, in some embodiments, housing 105 may include all of part of control device 160. Other configurations are possible.

Acoustic transducer 110 is adapted, at a minimum, to receive an acoustic signal. In one embodiment, acoustic transducer 110 is adapted to transmit an acoustic signal and to receive an acoustic “echo” produced by the transmitted acoustic signal. In another embodiment, acoustic transducer 110 receives an acoustic signal that has been transmitted or scanned by a separate device. Beneficially acoustic transducer 110 receives an acoustic signal that interrogates a three-dimensional volume of interest. In one embodiment, acoustic transducer 110 may include a two-dimensional acoustic transducer array that interrogates a three dimensional volume. In another embodiment, acoustic transducer 110 may include a one-dimensional acoustic transducer array that interrogates a scan plane at any one instant, and may be mechanically “wobbled” or electronically steered in a direction perpendicular to the scan plane to interrogate a three-dimensional volume of interest.

In one embodiment, acoustic imaging device 100 may be provided without an integral acoustic transducer 110, and instead may be adapted to operate with one or more varieties of acoustic transducers which may be provided separately.

Acoustic (e.g., ultrasound) signal processor 120 processes a received acoustic signal to generate data pertaining to a volume from which the acoustic signal is received.

Processor 140 is configured to execute one or more software algorithms in conjunction with memory 150 to provide functionality for acoustic imaging apparatus 100. In one embodiment, processor executes a software algorithm to provide a graphical user interface to a user via display device 130. Beneficially, processor 140 includes its own memory (e.g., nonvolatile memory) for storing executable software code that allows it to perform various functions of acoustic imaging apparatus 100. Alternatively, the executable code may be stored in designated memory locations within memory 150. Memory 150 also may store data in response to the processor 140.

Control device 160 provides a means for a user to interact with and control acoustic imaging apparatus 100.

Although acoustic imaging device 100 is illustrated in FIG. 1 as including processor 140 and a separate acoustic signal processor 120, in general, processor 140 and acoustic signal processor 120 may comprise any combination of hardware, firmware, and software. In particular, in one embodiment the operations of processor 140 and acoustic signal processor 120 may be performed by a single central processing unit (CPU). Many variations are possible consistent with the acoustic imaging device disclosed herein.

In one embodiment, processor 140 is configured to execute a software algorithm that provides, in conjunction with display device 130, a graphical user interface to a user of acoustic imaging apparatus 100.

Input/output port(s) 180 facilitate communications between processor 140 and other devices. Input/output port(s) 180 may include one or more USB ports, Firewire ports, Bluetooth ports, wireless Ethernet ports, custom designed interface ports, etc. In one embodiment, processor 140 receives one or more control signals from control device 160 via an input/output port 180.

Acoustic imaging apparatus 100 will now be explained in terms of an operation thereof. In particular, an exemplary operation of acoustic imaging apparatus 100 in conjunction with a nerve block procedure will now be explained.

Initially, a user (e.g., an anesthesiologist or an anesthesiologist's assistant) adjusts acoustic imaging apparatus 100 to interrogate a volume of interest within the patient's body. In particular, if a procedural device (e.g., a needle) is to be injected into a patient's nerve, the user adjusts acoustic transducer 110 to scan an acoustic signal through a volume of the patient's body that includes the part of the body (e.g., a nerve) where the needle is to be injected. In an embodiment where acoustic transducer 110 includes a 2D transducer array, it outputs 3D image volume data. In an embodiment where acoustic transducer 110 includes a 1D transducer array, at each instant in time acoustic transducer 110 outputs 2D image data representing a thin (e.g., 1 mm thick) slice of the volume of interest. In that case, the 1D array may be scanned or “wobbled” to generate volumetric data for an entire volume of interest in a fixed time interval.

Acoustic imaging apparatus 100 processes the received acoustic signal and identifies the procedural device (e.g., a needle) and its current location and orientation. Beneficially, acoustic imaging apparatus 100 may determine the trajectory of the procedural device.

In one embodiment, processor 140 executes a feature recognition algorithm to determine the location of the procedural device (e.g., a needle). Beneficially, the entire extent of an area occupied by the procedural device is determined. The feature recognition algorithm may employ one or more known features of the procedural device, including its shape (e.g., linear), its length, its width, etc. These features may be pre-stored in memory 150 of acoustic imaging apparatus 100 and/or may be stored in acoustic imaging apparatus 100 by a user in response to an algorithm executed by processor 140 and control device 160. In one embodiment at least a portion of the procedural device (e.g., the tip of the needle) may be coated with an ecogenic material that facilitates its recognition.

In another embodiment, acoustic imaging apparatus 100 generates and displays one or more images of the scanned volume to a user. The user may then employ control device 160 to manually identify the procedural device within the displayed image(s). For example, the user may manipulate a trackball or mouse to outline or otherwise to demarcate the boundaries of the procedural device in the displayed image(s). Processor 140 receives the user's input and determines the location of the procedural device. Again, in one embodiment at least a portion of the procedural device (e.g., the tip of the needle) may be coated with an ecogenic material that facilitates its recognition.

Then, in one embodiment, acoustic imaging apparatus 100 determines a first plane perpendicular to an orientation of the procedural device. For example, when the procedural device is a needle, then acoustic imaging apparatus 100 may determine the first plane as the plane that is perpendicular to a line extending through the length (long dimension) of the body of the needle at the tip of the needle. In another arrangement, acoustic imaging apparatus 100 may determine the first plane as the plane that is perpendicular to the trajectory of the procedural device at the periphery of the procedural device (e.g., the trajectory at the tip of the needle).

Then, in one embodiment, acoustic imaging apparatus 100 determines a second plane that is perpendicular to the first plane. Beneficially, the second plane may be selected such that it extends in parallel to a direction along with a body part of interest (e.g., a nerve) extends. However, other orientations of the second plane are possible. Indeed, in a beneficial embodiment, acoustic imaging apparatus 100 allows a user to select or change the second plane. After the first and second planes are determined, there is only one third plane which is perpendicular to both the first and second planes, and so the third plane can be determined from the first and second planes.

Acoustic imaging apparatus 100 then displays some or all of the first, second, and third planes via display device 130.

This can be better understood by reference to FIG. 2 which illustrates an exemplary arrangement of three planes with respect to a procedural device (e.g., a needle) 10 and a body part (e.g., a nerve) 20 toward which the procedural device is being directed. As seen in FIG. 2, a first plane 210 is perpendicular to an orientation of procedural device 10 (e.g., a needle) along the trajectory direction D. Second plane 220 is perpendicular to first plane 210 and extends in parallel to a direction along with nerve 20 extends. Third plane 230 is perpendicular to both the first and second planes 210 and 220 and cuts through a cross section of nerve 20.

FIG. 3A illustrates a display of the three planes shown in FIG. 2 according to a first example. The display shown in FIG. 3A may be displayed by display device 130 of acoustic imaging apparatus 100. Image 310 illustrates a two-dimensional view of first plane 210, image 320 illustrates a two-dimensional view of second plane 220, and image 330 illustrates a two-dimensional view of third plane 230 of FIG. 2. As noted above, in some embodiments acoustic imaging apparatus 100 may display less than all three of these planes.

In the example illustrated in FIG. 3A, the trajectory of needle 10 is offset slightly from nerve 20 so that its current trajectory will cause it to miss nerve 20. By means of this display, a user can easily recognize the problem and adjust the trajectory of the needle 10 so that it will intercept the nerve 20 at the desired location and angle.

FIG. 3B illustrates a display of the three planes shown in FIG. 2 according to a second example. As in FIG. 3A, in FIG. 3B image 310 illustrates a two-dimensional view of first plane 210, image 320 illustrates a two-dimensional view of second plane 220, and image 330 illustrates a two-dimensional view of third plane 230 of FIG. 2. Again, as noted above, in some embodiments acoustic imaging apparatus 100 may display less than all three of these planes.

In the example illustrated in FIG. 3B, the trajectory of needle 10 is such that it will penetrate nerve 20. By means of this display, a user can easily guide the needle 10 so that it will intercept the nerve 20 at the desired location and angle.

FIG. 4 illustrates a flowchart of a method of three dimensional acoustic imaging for medical procedure guidance by an acoustic imaging apparatus, such as acoustic imaging apparatus 100 of FIG. 1.

In a first step 410, an acoustic signal that interrogates a volume of interest is received by an acoustic transducer.

In a step 420, it is determined whether or not a user has selected a view to be displayed by the acoustic imaging apparatus. If so, then the process proceeds to step 460 as discussed below. Otherwise, the process proceeds to step 430.

In step 430 the acoustic imaging apparatus determines the location of a procedural device within the interrogated volume of interest. As described above, this can be done automatically using feature recognition and predetermined characteristics of the procedural device which may be stored in the acoustic imaging apparatus or entered into memory in the acoustic imaging apparatus by a user. Alternatively, the location of a procedural device can be determined with user assistance in identifying the procedural device within a displayed image.

In a step 440 the acoustic imaging apparatus determines a first plane that is perpendicular to an orientation of the procedural device. For example when the procedural device is a needle, then the acoustic imaging apparatus may determine a plane that is perpendicular to a line extending along the body of the needle at the tip of the needle. In another arrangement, the acoustic imaging apparatus may determine the first plane as the plane that is perpendicular to the trajectory of the procedural device at the periphery of the procedural device.

In an optional step 450, the acoustic imaging apparatus determines second and/or third planes that are perpendicular to the first plane. Beneficially, the second plane may be selected such that it extends in parallel to a direction along with a body part of interest (e.g., a nerve) extends. However, other orientations of the second plane are possible. After the first and second planes are determined, there is only one third plane which is perpendicular to both the first and second planes, and so the third plane can be determined from the first and second planes. In a case where only the first plane is to be displayed, in some embodiments step 450 may be omitted.

Where the user has selected a view to be displayed in step 420, then in a step 460 the acoustic imaging apparatus determines planes to be displayed for the user selected view. In one arrangement, the acoustic imaging apparatus determines the first plane that is perpendicular to an orientation of the procedural device, and the user then selects a desired second plane in step 420 that is perpendicular to the first plane. Alternatively, the user may select any of all of the plane(s) to be displayed.

In a step 470, the acoustic imaging apparatus 100 displays some or all of the first, second, and third planes to a user.

The process repeats so that the views of the planes are continuously updated as the procedural device is moved. In one embodiment, the plane views may be updated more than five times per second. In another embodiment, plane views may be updated more than 20 times per second, and beneficially, 30 times per second.

While preferred embodiments are disclosed herein, many variations are possible which remain within the concept and scope of the invention. For example, while for ease of explanation the examples described above have focused primarily on the application of regional anesthesiology, the devices and methods disclosed above may be applied to a variety of different contexts and medical procedures, including but not limited to procedures involving vascular access, RF ablation, biopsy procedures, etc. Such variations would become clear to one of ordinary skill in the art after inspection of the specification, drawings and claims herein. The invention therefore is not to be restricted except within the spirit and scope of the appended claims.

What is claimed is:

1. An acoustic imaging apparatus (100), comprising:
an acoustic signal processor (120) adapted to process an acoustic signal that is scanned to interrogate a volume of interest and is received by an acoustic transducer;
a display device (130) for displaying images in response to the processed acoustic signal;
a control device (160) that is adapted to allow a user to control at least one operating parameter of the acoustic imaging apparatus (100); and
a processor (140) configured to determine a location of a procedural device within the interrogated volume from the processed acoustic signal,
wherein the acoustic imaging apparatus (100) is configured to display on the display device (130) a first view of a first plane perpendicular to an orientation of the procedural device.

2. The acoustic imaging apparatus (100) of claim 1, wherein the display device (130) further displays a second view of a second plane perpendicular to the first plane.

3. The acoustic imaging apparatus (100) of claim 2, wherein the display device (130) further displays a third view of a third plane perpendicular to the first plane and to the second plane.

4. The acoustic imaging apparatus (100) of claim 3, wherein the display device (130) displays the first view, the second view, and the third view at a same time as each other.

5. The acoustic imaging apparatus (100) of claim 1, wherein the processor is configured to execute a feature recognition algorithm to determine the location of the procedural device within the interrogated volume from the processed acoustic signal.

6. The acoustic imaging apparatus (100) of claim 1, wherein the display device (130) is configured to display one or more images of the interrogated volume to a user, and the processor (140) is configured to receive an input from a user via the control device (160) identifying the procedural device within the displayed one or more images of the interrogated volume.

7. The acoustic imaging apparatus (100) of claim 1, wherein the acoustic imaging apparatus (100) is configured to identify the procedural device by identifying an echogenic material coated on at least a part of the procedural device.

8. The acoustic imaging apparatus (100) of claim 1, wherein the acoustic imaging apparatus (100) is configured to receive from the control device (160) an input from a user indicating a first user-selected plane to be displayed and in response thereto, to display on the display device (130) a view of the first user-selected plane.

9. The acoustic imaging apparatus (100) of claim 8, wherein the acoustic imaging apparatus (100) is further configured to display on the display device (130) a view of a second user-selected plane perpendicular to the first user-selected plane.

10. The acoustic imaging apparatus (100) of claim 1, wherein the acoustic imaging apparatus (100) is configured to continuously update the first and second views as the an orientation of the procedural device changes over time.

11. A method of three dimensional acoustic imaging for medical procedure guidance, comprising:
receiving (410) an acoustic signal that is scanned to interrogate a volume of interest;
determining (430) a location of a procedural device within the interrogated volume from the acoustic signal; and
displaying (470) on a display device (130) a first view of a first plane perpendicular to an orientation of the procedural device.

12. The method of claim 11, further comprising displaying (470) a second view of at least one plane perpendicular to the first plane.

13. The method of claim 12, further comprising displaying (470) on the display device (130) a third view of a third plane perpendicular to the first plane and to the second plane.

14. The method of claim 13, further comprising displaying the first view, the second view, and the third view at a same time as each other.

15. The method of claim 11, wherein determining (430) the location of the procedural device within the interrogated volume from the acoustic signal comprises executing a feature recognition algorithm.

16. The method of claim 11, wherein determining (430) the location of the procedural device within the interrogated volume from the acoustic signal comprises:
displaying via a display device (130) one or more images of the interrogated volume to a user; and
receiving an input from a user via a control device (160) identifying the procedural device within the displayed one or more images of the interrogated volume.

17. The method of claim 11, wherein determining (430) the location of the procedural device within the interrogated volume from the acoustic signal identifying an echogenic material coated on at least a part of the procedural device.

18. The method of claim 11, further comprising:
receiving (420) from a user via a control device (160) an indication of a first user-selected plane to be displayed; and
displaying (470) on the display device (130) a view of the first user-selected plane.

19. The method of claim 18, further comprising displaying (470) on the display device (130) a view of a second user-selected plane perpendicular to the first user-selected plane.

20. The method of claim 11, further comprising continuously updating the first and second views as an orientation of the procedural device changes over time.
